(19) United States
(12) Patent Application Publication
(10) Pub. No.: US 2023/0400458 A1
(43) Pub. Date: Dec. 14, 2023

(54) METHOD FOR NEUTRALIZING BIOTIN INTERFERENCE IN BINDING ASSAYS

(71) Applicant: Siemens Healthcare Diagnostics Products GmbH, Marburg (DE)

(72) Inventors: Herbert SCHWARZ, Lohra (DE); Norbert ZANDER, Marburg (DE); Carolin BALOUSCHEK, Coelbe (DE); Matthias EHM, Lahntal/Sterzhausen (DE)

(21) Appl. No.: 18/203,167

(22) Filed: May 30, 2023

(30) Foreign Application Priority Data
Jun. 10, 2022 (EP) 22178270.9

(51) Int. Cl. G01N 33/543

(52) U.S. Cl. CPC G01N 33/54393

(57) ABSTRACT

A method is provided herein for neutralizing biotin interference in a binding assay for detection of an analyte in a sample, wherein the binding assay comprises contacting the sample at least with a biotinylated component and with a biotin-binding component, wherein the sample is contacted with an adsorbent substance before or during the contacting with the biotinylated component and with the biotin-binding component. A coated solid-phase system for neutralizing biotin interference in a binding assay for detection of analyte in a sample is also provided herein.

### CROSS-REFERENCE TO RELATED APPLICATION(S)

The present application claims priority under 35 U.S.C. § 119 to European Patent Application No. 22178270.9, filed 10 Jun. 2022, the entire contents of which are incorporated herein by reference.

### FIELD

One or more example embodiments of the present invention relate to a method for neutralizing biotin interference in a binding assay for detection of an analyte in a sample, wherein the sample is contacted with an adsorbent substance. In another embodiment, the invention further relates to a coated solid-phase system for neutralizing biotin interference in a binding assay for detection of an analyte in a sample.

### BACKGROUND

Biotin, which is also referred to as vitamin B7 or vitamin H, is a water-soluble vitamin which plays an important role in the metabolism of fatty acids, carbohydrates and proteins and in epigenetic gene regulation. The human organism cannot itself synthesize biotin and is dependent on absorption through food. According to the Deutsche Gesellschaft für Ernährung (German society for nutrition), the appropriate intake of biotin for children and adults, including those who are pregnant and nursing, is estimated at from 30 to 60 micrograms per day. There are numerous food supplements on the market that cover the aforementioned daily biotin requirement, but there are also those which are in a distinctly higher dosage range of, for example, ten milligrams per day. In general, biotin-containing food supplements are used to assist metabolism and neurological function and to promote healthy skin, hair and nails.

However, the use of biotin extends well beyond the therapeutic and cosmetic purposes mentioned. The highly selective and stable interaction between biotin and avidin or streptavidin is widely used in biotechnological and clinical laboratory techniques and has, for example, been used for decades in a variety of biochemical, immunological and molecular biology techniques, including sensitive and precise binding assays.

Owing to their bioanalytical capability, in vitro diagnostic binding assays are widespread in clinical laboratories. Their complete automatability provides a number of advantages, such as rapid performance of the assays and high sensitivity and precision. In binding assays, analyte-specific binding partners which specifically interact with the analyte to be determined act as detection molecules. Typical binding assays are immunoassays, which comprise the recognition and detection of an analyte through antigen-antibody binding. Depending on the configuration of the assay, either the antigen or the antibody can be the analyte to be detected. In such immunoassays, the streptavidin-biotin system is, for example, used for the purposes of enrichment, removal or signal enhancement. For example, streptavidin-coated magnetic particles on a solid phase can be used as a means for specific binding of biotinylated antibodies to which the analyte to be detected has bound, in order to enrich the analyte from the liquid phase followed by detection of said analyte.

When carrying out such binding assays comprising a biotin-streptavidin interaction, an elevated biotin concentration in the sample can cause significant disruptive effects, since the sample-intrinsic biotin competes with biotinylated assay components for binding sites on streptavidin. In sandwich assay formats, the signal intensity measured is typically directly proportional to the analyte concentration. Sample-intrinsic biotin competes with the biotinylated binding partners for binding to streptavidin-coupled signal components, thus weakens the signal intensity and ultimately produces falsely low assay results. In competitive assay formats, the signal intensity is typically inversely proportional to the analyte concentration. Here too, sample-intrinsic biotin influences the signal intensity and results in a falsely high assay result. In general, biotin interferences lead to falsely low results with respect to the analyte concentration.

In the case of laboratory tests for patients who ingest biotin, falsely high or falsely low analyte concentrations, depending on the assay format, may thus be measured if the technique used involves the principle of a streptavidin-biotin assay. Unidentified problems caused by biotin that affect laboratory tests thus mean a high risk of delayed diagnoses, false diagnoses and unnecessary treatments.

Methods for reducing biotin interference are known from the prior art, said methods providing pretreatment of the sample with streptavidin-coated magnetic beads (Yang et al., 2020, Clinica Chimica Acta, 505, 130-135) or focusing on the use of microparticles provided with streptavidin (Bowen et al., 2019, Clinical Biochemistry, 74, 1-11). These approaches typically require a relatively long incubation and a subsequent time-consuming removal of the capture units, for example by means of centrifugation, before further assay steps can be carried out.

Therefore, methods for neutralizing biotin interference in biotin-streptavidin-based binding assays are known, in which the sample is contacted with a biotin-binding substance before the contacting with the biotinylated component and with the biotin-binding component.

### SUMMARY

Therefore alternative methods and means are provided herein that make it possible to efficiently neutralize possible biotin interference in binding assays.

In an example embodiment, the problem is essentially solved, in a binding assay for detecting an analyte in a sample, by contacting the sample with an adsorbent substance before or during the contacting with a biotinylated component and with a biotin-binding component, wherein the adsorbent substance has a porous structure having a total surface area (S_{Bet}) of from 300 to 6500 m² g⁻² and has at least macropores, mesopores and micropores, wherein the volume of the micropores (V_{mic}) is from 0.15 to 0.75 cm³ g⁻².

This has the effect of binding any biotin present in a sample in an efficient and hugely time-saving manner and of thus neutralizing the disruptive effects thereof on the assay result. The assay steps based on biotin-avidin/streptavidin interaction can then be carried out without any influence by free biotin. As a result, falsely low or falsely high results, depending on the assay format, can be virtually ruled out. This significantly increases the reliability of the assay procedure. Furthermore, risk assessment by biotin titer determination, which has hitherto usually been carried out, is no longer necessary.

### DETAILED DESCRIPTION

In one embodiment the present invention thus provides a method for neutralizing biotin interference in a binding assay for detection of an analyte in a sample, wherein the binding assay comprises contacting the sample at least with a biotinylated component and with a biotin-binding component and wherein the sample is contacted with an adsorbent substance before or during the contacting with the biotinylated component and with the biotin-binding component, wherein the adsorbent substance has a porous structure having a total surface area (S_{Bet}) of from 300 to 6500 m² g⁻¹ and has at least macropores, mesopores and micropores, wherein the volume of the micropores (V_{mic}) is from 0.15 to 0.75 cm³ g⁻¹.

In an example embodiment according to the present invention, the adsorbent substance has a porous structure. Porosity and pore structure is preferably characterized by the parameter of total surface area (S_{Bet}), which can be within the range from 300 to 6500 m² g⁻¹. In specific embodiments, the total surface area can be from 300 to 600 m² g⁻¹ or from 400 to 3500 m² g⁻¹ or from 400 to 5000 m² g⁻¹. In further specific embodiments, the total surface area can be 300, 400, 500, 600, 700, 800, 900, 1000, 1500, 2000, 2500, 2600, 2700, 2800, 2900, 3000, 3100, 3200, 3300, 3400, 3500, 4000, 4500, 5000, 5500, 6000 or 6500 m² g⁻¹ or any value between the values stated. Preference is given to a total surface area of from 2000 to 3500 m² g⁻¹.

Furthermore, the substance has at least macropores, mesopores and micropores. A “macropore” is a pore having a diameter of greater than 50 nm. A “mesopore” is a pore having a diameter of from 2 to 50 nm. A “micropore” is a pore having a diameter of less than 2 nm. Biotin is typically adsorbed in the micropore range, and so the volumes of the micropores are an indicator of the adsorption capacity of the substance.

The volume of the micropores (V_{mic}) of the adsorbent substance according to the invention is furthermore from 0.15 to 0.75 cm³ g⁻¹. In specific embodiments, the volume of the micropores can be from 0.15 to 0.25, from 0.2 to 0.5, from 0.3 to 0.75, from 0.15 to 0.2 or from 0.25 to 0.65 cm³ g⁻¹. The volume of the micropores can furthermore have a value of 0.15, 0.2, 0.3, 0.35, 0.4, 0.45, 0.5, 0.55, 0.6, 0.65, 0.7 or 0.75 cm³ g⁻ or any intermediate value.

An adsorbent substance according to an embodiment of the invention is distinguished by the fact that it enriches biotin at its surface, i.e., at the interface between the solid phase and the liquid phase. The adsorption may be physical adsorption or chemical adsorption, depending on the substance used. “Physical adsorption” is typically a physical process in which molecules remain adherent on the surface of another substance and are enriched on the surface of said substance. The forces causing the adherence are van der Waals forces. Typically, the molecules arrive at the interface as a result of their undirected, thermally driven molecular motion. In the case of “chemical adsorption”, the molecules are bound on the surface of a solid by chemical bonds and also modify the solid at the same time, i.e., form covalent bonds, for example, with said solid.

A preferred adsorbent substance is activated carbon, a metal-organic framework (MOF), zeolite, clay, a porous organic polymer (POP), hydrotalcite, an organic-inorganic hybrid, a porous metal oxide, a lithium zirconate or a mesoporous silica material (MSM).

Activated carbon predominantly consists of carbon having a highly porous structure. The pores are typically open-pored and connected to one another. The total surface area is generally between 300 and 3000 m² g⁻¹, for example 2900 or 3000 m² g⁻¹. The density of activated carbon is typically within the range from 0.2 to 0.6 g/cm³. Activated carbon has not only micropores, mesopores and macropores, but also submicropores of a size of <0.4 nm. The macropores and mesopores are typically the access routes for gases or liquids into the interior of the activated carbon and bring about the diffusion and mass-transfer processes into inner regions of the substance. Adsorption occurs at the surface of the micropores.

Activated carbon can be used in all useful forms and variants known to a person skilled in the art. For example, activated carbon can be used in tablet form. In particularly preferred embodiments, DOAC-Stop or DOAC-Remove activated carbon tablets are used. DOAC-Stop tablets, which are sold by Haematex for example, consist of specific activated carbon mixtures. DOAC-Remove activated carbon tablets, which are sold by Coachrom or 5-Diagnostics AG, likewise consist of specific activated carbon mixtures. Further forms of activated carbon which can be used in the context of the present invention include products having CAS No. 7440-44-0. Further details, including details of further forms of activated carbon that are likewise envisaged in the invention, are known to a person skilled in the art or can be found in suitable literature sources, for example Barnes et al., 2009, Carbon, 47, 1887-1895.

“Metal-organic frameworks (MOFs)” are microporous materials formed from inorganic building units and from organic molecules as connecting elements between the inorganic building units. Metal-organic frameworks are typically crystalline and can be characterized as coordination polymers or coordination networks having an open framework containing pores. Examples of MOFs, as can be used in the context of the present invention, are MOF-5, which consists of Zn₄O tetrahedrons to which 1,4-benzenedicarboxyl (BDC) organic ligands are attached, MOF-177 (with BTB), MOF-180 (with BTE), MOF-200 (with BBC), MOF-205 (with BTB and NDC), MOF-210 (with BTE and BPDC) or CD-MOFs, which use cyclodextrins as linkers and alkali metal cations as connectors. Further details are known to a person skilled in the art or can be found in suitable literature sources, for example Reddy et al., 2021, RSV Adv., 11, 12658.

The term “zeolite” relates to crystalline aluminosilicates. The composition of the zeolite group of substances is typically indicated by Mⁿ⁺_{x/n} [(AlO₂)⁻ₓ (SiO₂)_{y}]·z H₂O, where n refers to the charge of the cation M and can be 1 or 2. M is typically a cation of an alkali metal or alkaline earth metal. These cations are needed to compensate for the electric charge of the negatively charged aluminum tetrahedron; they are not incorporated into the main lattice of the crystal, but reside in voids of the lattice and can move within the lattice. z indicates the number of water molecules taken up by the crystal. The molar ratio of SiO₂ to AlO₂ or y/x in the molecular formula is referred to as a modulus. Zeolites thus consist of a microporous framework structure composed of AlO₄⁻ and SiO₄ tetrahedrons, the aluminum and silicon atoms being connected to one another by oxygen atoms. Depending on the structural type, this yields a structure of uniform pores and/or channels in which substances can be adsorbed. Examples of zeolites, as can be used in the context of the present invention, include MFI zeolites, CHA zeolites, LTA zeolites, zeolite A, zeolite X, zeolite L, ZSM 11, ZSM 5, or zeolite Y. Zeolites generally have a pore size in the micropore range of approx. 2 nm. Further details are known to a person skilled in the art or can be found in suitable literature sources, for example Reddy et al., 2021, RSV Adv., 11, 12658.

The term “clay” in the context of the present invention refers to phyllosilicates, which have a layered crystal structure composed of silicon and oxygen and also hydrogen and usually magnesium and aluminum. Further details are known to a person skilled in the art or can be found in suitable literature sources, for example Reddy et al., 2021, RSV Adv., 11, 12658.

“Porous organic polymers (POPs)” are a class of multidimensional porous network materials formed via strong covalent bonds between various organic building blocks having different geometries and topologies. POPs can generally be divided into two categories, amorphous and crystalline POPs, on the basis of their degree of long-range order. Amorphous POPs include mainly hypercrosslinked polymers (HCPs), polymers of intrinsic microporosity (PIMs), conjugated microporous polymers (CMPs) and porous aromatic frameworks (PAFs), whereas crystalline POPs include covalent organic frameworks (COFs). They are of a low weight, have a well-developed inherent porosity, exhibit high stability, are preconfigurable, and have adjustable structures and functions. Advantageously, it is possible to design their porous structure, pore size, specific surface regions and functions and to influence them by insertion of specific functional building blocks. Further details are known to a person skilled in the art or can be found in suitable literature sources, for example Reddy et al., 2021, RSV Adv., 11, 12658.

“Hydrotalcite” is a mineral from the class of carbonates. It crystallizes in a trigonal crystal system having the chemical composition Mg₆Al₂[(OH)₁₆|CO₃]·4H₂O and usually develops transparent, tabular crystals of up to about 4 mm in size with crystal surfaces having a silky luster or waxy luster. Further details are known to a person skilled in the art or can be found in suitable literature sources, for example Reddy et al., 2021, RSV Adv., 11, 12658.

An “organic-inorganic hybrid” is a class of materials, the structure of which comprises both organic and inorganic units that interact with one another at the molecular level. Said materials are divided into two classes on the basis of the interaction between organic and inorganic components. In class I, organic and inorganic components are embedded, and there are weak interactions between them, such as hydrogen bonds, van der Waals interactions, n-n interactions or weak electrostatic interactions. In class II, these two components are connected to one another by strong covalent or coordinate bonds. Organic-inorganic hybrid polymers can, for example, be obtained by sol-gel methods, self-assembly processes or assembly or dispersion of nano-building blocks. Examples include core-shell nanocomposites (CSNs), polystyrene-coated Fe₂O₃ particles or interpenetrating networks (IPNs). Further details are known to a person skilled in the art or can be found in suitable literature sources, for example Reddy et al., 2021, RSV Adv., 11, 12658.

A “porous metal oxide (MO)” is a compound formed between a metal and oxygen. Owing to structure-conducting measures based on carbon or silica matrices in mesoporous form, there are now porous metal oxides of differing character, such as TiO₂, ZrO₂, Al₂O₃, V₂O₅, MoO₃, Fe₂O₃, MgAl₂PO₄, MgO₂, CeO₂ or MnO₂. Further details are known to a person skilled in the art or can be found in suitable literature sources, for example Reddy et al., 2021, RSV Adv., 11, 12658.

Lithium zirconate can likewise form porous structures. Further details are known to a person skilled in the art or can be found in suitable literature sources, for example Reddy et al., 2021, RSV Adv., 11, 12658.

“Mesoporous silica materials (MSMs)” are distinguished by a high symmetry of the pore arrangement, narrow pore-size distributions and surface areas of up to 1000 m²/g. The pore walls of MSMs typically consist of pure, amorphous SiO₂. Examples of MSMs include the M41 S family, for example MCM41, which has hexagonally arranged cylindrical pores. A further example is the SBA family, the members of which have a MCM41-comparable hexagonal pore structure and are distinguished by high hydrothermal stability because of their high pore-wall thickness and also have combined microporosity and mesoporosity because of the PO side chains of the polymeric templates, for example SBA15. Further classes include KIT-6 and MCM-48. Further details are known to a person skilled in the art or can be found in suitable literature sources, for example Reddy et al., 2021, RSV Adv., 11, 12658.

The sample can be contacted with the adsorbent substance by mixing the sample with the substance in the form of a powder, granular material or microfiber mixture to form a suspension. As a result, the biotin from the sample is attached to the adsorbent substance and thus quantitatively withdrawn from the sample, which can be evaluated in a binding assay without any further interference by biotin. The sample is mixed with the adsorbent substance before detection steps for an analyte are carried out.

The sample can then be removed from the suspension of the adsorbent substance before the subsequent contacting with the biotinylated component and with the biotin-binding component. The removal can, for example, be achieved by means of centrifugation or filtration.

Alternatively, the sample can be contacted with the adsorbent substance by contacting the sample with a solid phase coated with the substance.

In specific embodiments, the adsorbent substance as described above can have been applied to a solid phase. The term “solid phase” in the context of the invention refers to an object which consists of porous and/or nonporous, water-insoluble material and can take different forms, for example a vessel, a pipette, a small tube, a microtitration plate, a bead, a microparticle, rods, a strip, filter or chromatography paper, etc. Generally, the surface of the solid phase is hydrophilic. The solid phase can consist of different materials, for example inorganic and/or organic materials, synthetic materials, naturally occurring materials and/or modified naturally occurring materials. Examples of solid-phase materials are polymers, for example cellulose, nitrocellulose, cellulose acetate, polyvinyl chloride, polyacrylamide, crosslinked dextran molecules, agarose, polystyrene, polyethylene, polypropylene, polymethacrylate or nylon; ceramic; silicate; glass; metals, for example magnetizable metals such as iron, or precious metals such as gold and silver; magnetite; mixtures or combinations of same; etc.

In preferred embodiments, the solid phase can be a filter, the surface of a liquid container, a pipette tip, or a particle composed of latex, Sepharose, agarose, plastic, glass, protein, alginate, silicate or metal.

In particular embodiments, the solid phase is a magnetizable particle which, by means of application of a magnetic field, can be withdrawn from a mixture or suspension or be immobilized at a particular position.

A solid phase is coated with an adsorbent substance in such a way that the porosity of the substance is maintained, thus making attachment of biotin possible. Depending on the class of substance, it is necessary to take into account different coating thicknesses and different forms of coating. Such details are known to a person skilled in the art and can be found in suitable literature sources, for example Kim et al., 2016, Scientific Reports, 6, 21182 or Rocher et al., 2008, Water Res, 42(4-5), 1290-8. To reduce the amount of biotin, the described solid phase is contacted with the sample. Here, biotin can be attached to the solid phase via the pores present and relevant interactions. As a result, it has been quantitatively withdrawn from the sample, which can be evaluated in a binding assay without any further interference by biotin.

In a further preferred embodiment of the method, the adsorbent substance has been pretreated with one or more blocking proteins or blocking peptides, preferably with one or more blocking proteins from the group consisting of dextran, albumin, polygeline and milk protein.

Alternatively or additionally, the adsorbent substance can have been coated with surface blockers. A “surface blocker” in the context of the present invention is understood to mean a molecule which saturates free binding sites on surfaces of adsorbent substances and thus helps to avoid analytes and assay components being captured on surface structures of the substance. This prevents distortion of an assay result. Examples of surface blockers which can be used in the context of the invention include albumin, for example BSA or HSA, dextrans, i.e., branched polysaccharides of high molecular weight, for example aminodextran, parylene, polyxylene, Sepharose, i.e., a processed form of the polymer agarose, or polygeline, i.e., a polymer composed of urea and hydrolyzed gelatin, for example Haemaccel. Further surface blockers are casein or milk protein or casein-based reagents, for example the commercially available Blocking Solution from Candor, which is based on highly pure casein which has been altered by chemical modifications. The result is a spectrum of casein fragments of different sizes. Further possibilities are peptide combinations which have been chemically modified and can thus penetrate into small gaps on the surface, for example the product SmartBlock. In further embodiments, coating with a surface blocker can be followed by additional stabilization with sugar solutions or commercial solutions such as Liquid Plate Sealer. These stabilizers surround the surface blockers and prevent their degradation and removal.

It is further preferred that the adsorbent substance is provided in the form of polymer-coated substance beads having a pore size with permeability for biomolecules of a mass <40 kDa.

Alternatively, the adsorbent substance can be provided in the form of polymer-coated beads or particles. For example, it is possible to produce particles based on acrylic acid, methacrylic acid, methyl methacrylic acid, p-vinylbenzoic acid, 2-methyl butanediol acid, 1-ethyl-4-vinylbenzene, styrene, 4-vinylpyridine, 1-vinylimidazole, acrylamide, methacrylamide, alginate or silicate. The particles can furthermore be provided with magnetizable components, for example iron oxide. It is preferred that the particles have a pore size having a permeability for biomolecules of a mass <40 kDa, for example kDa, 30 kDa, 25 kDa, 20 kDa, 15 kDa, 10 kDa, 5 kDa, 2 kDa, 1 kDa, 0.5 kDa, 0.25 kDa or smaller. The porosity of the particles can be controlled via the nature of the substance. Furthermore, it is possible to use matrix structures for generation of pores, especially when using metal oxides. Such details are known to a person skilled in the art and can be found in suitable literature sources, for example Kim et al., 2016, Scientific Reports, 6, 21182 or Rocher et al., 2008, Water Res, 42(4-5), 1290-8.

The term “biotin interference”, as used herein, refers to the presence of biotin in a binding assay for detection of an analyte in a sample, the biotin not being present as a functional component of the binding assay, but originating from the sample to be tested. As a result, this free biotin interferes with the interaction forming the basis of the binding assay, the interaction of a biotinylated assay component with a biotin-specific binding partner, for example streptavidin or structurally or functionally similar components, and influences or distorts the result of the binding assay because it binds to the biotin-specific binding partner and thereby blocks it for detection reactions.

The term “binding assay” essentially includes methods for detecting analytes in which an analyte-containing sample is contacted with one or more analyte-specific binding partners and the resultant complex(es) composed of analyte and analyte-specific binding partner(s) is/are quantitatively determined. A classic binding assay format is, for example, the so-called sandwich assay, in which an analyte is bound by a capture binding partner and a detection binding partner. In addition, there are further numerous binding assay formats well-known to a person skilled in the art (competitive binding assays, heterogeneous binding assays, homogeneous binding assays, etc.). Analyte-specific binding partners preferably used in the binding assays are antibodies, antibody fragments, affimers or aptamers.

In the binding assays improved by the invention, the sample is contacted with at least a biotinylated component and with a biotin-binding component. Depending on the assay format, the biotinylated component and the biotin-binding component are coordinated with one another in such a way that the interaction between biotin and biotin-binding partner accomplishes enrichment or removal of the analyte or signal enhancement or some other function appropriate for the assay format. To this end, the biotin-binding component and/or the biotinylated component can contain a peptide, a protein, an antibody, an antibody fragment, or an affimer or aptamer.

A “biotinylated component” is thus to be understood to mean an assay component which comprises a functional unit, for example (i) a binding partner from the group consisting of peptide, protein, antibody, antibody fragment, affimer and aptamer, (ii) a component of a signal-forming system and/or (iii) a solid phase (e.g., particle), to which one or more biotin molecules have been directly or indirectly coupled.

A “biotin-binding component” is to be analogously understood to mean an assay component which has binding sites for the specific binding of biotin. Preferably, the “biotin-binding component” is a functional unit, for example (i) a binding partner from the group consisting of peptide, protein, antibody, antibody fragment, affimer and aptamer, (ii) a component of a signal-forming system and/or (iii) a solid phase (e.g., particle), to which one or more biotin-binding molecules have been directly or indirectly coupled. Preferred biotin-binding molecules are avidin, streptavidin, tamavidin 1 or 2, shwanavidin, and/or rhizavidin, bradavidin, burkavidin, zebavidin, xenavidin and/or strongavidin.

It is further preferred that the biotin-binding component and/or the biotinylated component contains a peptide, a protein, an antibody, an antibody fragment, an affimer or an aptamer.

The application of the components described is not restricted to the standard scenarios discussed in detail above, but can be varied and adapted depending on the requirements, analyte and desired measurement result.

In an additional, preferred embodiment, the biotinylated component contains a first component of a signal-forming system and the biotin-binding component contains a second component of the signal-forming system, wherein the first and the second component of the signal-forming system interact in such a way that a detectable signal is formed if the first and the second component of the signal-forming system are brought into physical proximity with one another.

In a further preferred embodiment, the first component of the signal-forming system can be a chemiluminescent agent and the second component of the signal-forming system can be a photosensitizer or vice versa.

A “photosensitizer” is a substance, the absorption range of which is within the wavelength range of the irradiated light and acts as a photochemical converter, i.e., it transfers the light energy to a second molecule which has different absorption properties, but can react after the transfer of the light energy. Examples are, for instance, phthalocyanine. Furthermore, a system such as AlphaLISA, which uses luminescent oxygen channeling chemistry for detection, can be used. Further details can be found in, for example, Pulido-Olmo et al., 2017, Frontiers in Immunology, 8, Article 853.

A “chemiluminescent agent” is an agent which, after a chemical reaction, emits electromagnetic radiation within the ultraviolet light range or visible light range. Examples of chemiluminescent compounds are luminol or imidazole. In a broader sense, bioluminescent proteins are also included, for example luciferin, luciferase, green fluorescent protein (GFP), mCherry, mOrange, TagBFP, Cerulean, Citrine, mTurquoise, red fluorescent protein (RFP), yellow fluorescent protein (YFP) and derivatives thereof. Further chemiluminescent compounds which can be used in the context of the present invention include 6-FAM, HEX, TET, ROX, Cy2, Cy3, Cy5, Cy7, Texas Red or rhodamines, PerCP, Pacific Blue, APC, Alexa 405, 430, 488, 546, 559, 594, 633, 660, 674, 680, 700, Cascade Blue, TAMRA, Dabcyl, Black Hole Quencher, BHQ-1 or BHQ-2. Further details, including details of an assay format based on the elements mentioned, are known to a person skilled in the art or can be found in Pulido-Olmo et al., 2017, Frontiers in Immunology, 8, Article 853.

The sample to be analyzed is preferably a biological sample. The term “biological sample” generally refers to an animal body fluid, for example a vertebrate body fluid, preferably a mammalian body fluid, particularly preferably a human body fluid. All types of body fluids in which analytes can be potentially found and/or tested are included. Preference is given to, in particular, whole blood, blood plasma, blood serum, saliva, cerebrospinal fluid or lacrimal fluid.

In a further aspect, the application relates to the use of an adsorbent substance, wherein the substance has a porous structure having a total surface area (S_{Bet}) of from 300 to 6500 m² g⁻¹ and has at least macropores, mesopores and micropores, wherein the volume of the micropores (V_{mic}) is from 0.15 to 0.75 cm³ g⁻¹, for neutralizing biotin interference in a binding assay for detection of an analyte in a sample, wherein the binding assay comprises contacting the sample at least with a biotinylated component and with a biotin-binding component. The substance is preferably activated carbon, a metal-organic framework (MOF), zeolite, clay, a porous organic polymer (POP), hydrotalcite, an organic-inorganic hybrid, a porous metal oxide, a lithium zirconate or a mesoporous silica material (MSM). The components and elements of the binding assay correspond to the above-described components and elements.

The use of the adsorbent substances for neutralizing biotin interference ideally takes place before the contacting of samples and assay components, for example by preincubation/pretreatment of the sample with the adsorbent substance. The use according to the invention is, at the same time, not limited to specific assay formats or specific binding assays, but includes all biotin-based interaction schemes in which an elevated biotin level in the sample is (potentially) present or is suspected.

A further embodiment of the present invention is a coated solid-phase system suitable for neutralizing biotin interference in a binding assay for detection of an analyte in a sample.

The coated solid-phase system according to an embodiment of the invention is distinguished by the fact that (i) the coating comprises an adsorbent substance, wherein the substance has a porous structure having a total surface area (S_{Bet}) of from 300 to 6500 m² g⁻¹ and has at least macropores, mesopores and micropores, wherein the volume of the micropores (V_{mic}) is from 0.15 to 0.75 cm³ g⁻¹, wherein the substance is preferably activated carbon, a metal-organic framework (MOF), zeolite, clay, a porous organic polymer (POP), hydrotalcite, an organic-inorganic hybrid, a porous metal oxide, a lithium zirconate or a mesoporous silica material (MSM); and (ii) the solid phase is selected from the group consisting of filter, surface of a liquid container, pipette tip, and particle composed of latex, Sepharose, agarose, plastic, glass, protein, alginate, silicate or metal.

The coating can be applied as discussed further above. Typically, the coating is applied in such a way that the porous structure of the adsorbent substance is not impaired and the functional properties regarding the attachment of biotin are maintained.

The solid phase can, at the same time, be selected from all suitable materials. It is preferred to use materials and products which are in any case exposed to contact with the sample to be analyzed before carrying out or possibly while carrying out a binding assay or assay. For example, this can be a filter, the surface of a liquid container, or a pipette tip. Further materials include particles composed of latex, Sepharose, agarose, plastic, glass, protein, alginate, silicate or metal. Further details are known to a person skilled in the art and can be found in suitable literature sources, for example Kim et al., 2016, Scientific Reports, 6, 21182 or Rocher et al., 2008, Water Res, 42(4-5), 1290-8.

Although the present invention is described with respect to particular embodiments, this description is not to be construed in a limiting sense.

As used in this description and in the appended claims, the singular forms of “a” and “an” also include the respective plural forms, unless the context clearly dictates otherwise.

In connection with the present invention, the terms “approximately” and “about” refer to an interval of accuracy that a person skilled in the art understands to still ensure the technical effect of the feature in question. The term typically indicates a deviation from the indicated numerical value of ±20%, preferably ±15%, more preferably ±10% and even more preferably ±5%.

It is to be understood that the term “comprising” is not limiting.

If hereinafter a group is defined to comprise at least a certain number of embodiments, this is meant to also encompass a group which preferably consists of these embodiments only.

Furthermore, the terms “(i)”, “(ii)”, “(iii)” or “(a)”, “(b)”, “(c)”, “(d)” or “first”, “second”, “third”, etc. and the like in the description or in the claims are used for distinguishing between similar elements and not necessarily for describing a sequential or chronological order.

It is to be understood that the terms so used are interchangeable under appropriate circumstances and that the embodiments of the invention described herein can be used in a different order than described herein. In case the terms relate to steps of a technique, method or use, there is no time or time interval coherence between the steps, i.e., the steps may be carried out simultaneously or there may be time intervals of seconds, minutes, hours, days, weeks, etc., between such steps, unless otherwise indicated.

It is to be understood that this invention is not limited to the particular methods, protocols, etc., described herein, since they may vary. It is also to be understood that the terminology used herein is for the purpose of describing particular embodiments only, and is not intended to limit the scope of the present invention that will be limited only by the appended claims.

Unless defined otherwise, all technical and scientific terms used herein have the same meanings as commonly understood by a person of ordinary skill in the art.

The following examples and figures are provided for illustrative purposes. It is thus to be understood that the examples and figures are not to be construed as limiting. A person skilled in the art will clearly be able to envisage further modifications of the principles laid out herein.

### EXAMPLES

### Example 1: Neutralization of Biotin Interference in Accordance with the Invention in an F1+2 Immunoassay by Sample Pretreatment Using Activated Carbon Tablets

Samples of a pool of normal citrate plasma were spiked with 60, 150, 1700 or 2600 pmol/L F1+2 peptide (prothrombin fragment F1+2) and, in each case, with 0, 500, 1000 or 2000 ng/mL biotin (Sigma) and assayed on the Siemens Healthineers Atellica COAG360 coagulation analyzer using the INNOVANCE LOCI F1+2 assay (Siemens Healthineers), as described in the package insert.

The INNOVANCE LOCI F 1+2 assay comprises, inter alia, a first reagent containing a biotinylated first antibody having specificity for the F1+2 peptide (biotinylated assay component), a second reagent containing a second antibody having specificity for the immune complex composed of F1+2 peptide and first antibody, the second antibody being coupled to Chemibeads, and a third reagent containing streptavidin-coated Sensibeads (biotin-binding assay component). The reagents are mixed with the sample. In the reaction, the streptavidin-coated Sensibeads bind, via the biotin-streptavidin interaction, to the biotinylated antibody having specificity for the F1+2 peptide. If F1+2 peptide is present in the sample, the biotinylated first antibody binds thereto, and the second antibody binds in turn to the resultant immune complex composed of F1+2 peptide and first antibody. In this way, the Chemibeads and the Sensibeads are brought into physical proximity with one another, so that a chemiluminescence signal is formed and can be measured.

To neutralize biotin interference, 1.0 mL each of the above-described samples was pretreated with activated carbon. For this purpose, 1 tablet of DOAC-Stop (Haematex Research) was added to 1.0 mL of sample, followed by careful mixing. 10 minutes after the tablet had been added, the samples were centrifuged at 2500 g for 2-5 minutes. The plasma supernatant was carefully pipetted off.

The samples pretreated in this way were then automatically analyzed in the Siemens Healthineers Atellica COAG360 coagulation analyzer using the INNOVANCE LOCI F 1+2 assay.

Each sample was assayed in triplicate, and the mean of the measured F1+2 peptide concentrations was calculated. The standard deviation (STD) and the coefficient of variation (CV) were determined.

Table 1 shows the biotin interference in the INNOVANCE LOCI F1+2 assay in samples containing 0-2000 ng/mL biotin and the elimination of the biotin interference by the pretreatment of the samples with activated carbon. The described technique shows 101%-90% recovery of the treated samples in the INNOVANCE LOCI F1+2 assay for samples containing up to 2000 ng/mL biotin. In the absence of pretreatment, an increasing deviation from the starting value of the sample with no biotin added becomes apparent in samples with an increasing content of biotin. Just 500 ng/mL biotin lead to a deviation of up to 25.29% in relation to the starting value of the sample without biotin. Contents of 1000 ng/mL and 2000 ng/mL biotin in the sample exhibit very high interference and lead to a deviation of up to 62.25% and 98.76% from the starting value of the sample without biotin.

TABLE 1

| | Biotin | Activated | Measurement 1 | Measurement 2 | Measurement 3 | Mean | | | Deviation from |
|---|---|---|---|---|---|---|---|---|---|
| Sample | [ng/ml] | carbon added | pmol/L F1 + 2 | pmol/L F1 + 2 | pmol/L F1 + 2 | pmol/L F1 + 2 | STD | CV | starting value in % |
| Sample, | 0 | | 64.11 | 63.31 | 64.26 | 63.89 | 0.51 | 0.80% | 0 |
| 60 | 0 | x | 61.64 | 61.19 | 61.22 | 61.35 | 0.25 | 0.41% | 3.98 |
| pmol/L | 500 | | 53.84 | 55.05 | 54.85 | 54.58 | 0.65 | 1.19% | 14.58 |
| F1 + 2 | 500 | x | 57.92 | 57.91 | 57.82 | 57.88 | 0.06 | 0.10% | 9.41 |
| | 1000 | | 34.53 | 34.70 | 34.06 | 34.43 | 0.33 | 0.96% | 46.11 |
| | 1000 | x | 58.15 | 58.08 | 57.72 | 57.98 | 0.23 | 0.40% | 9.25 |
| | 2000 | | 0.85 | 1.31 | 1.31 | 1.16 | 0.27 | 22.96 | 98.19 |
| | 2000 | x | 57.77 | 56.99 | 57.10 | 57.29 | 0.42 | 0.74% | 10.34 |
| Sample, | 0 | | 150.93 | 150.08 | 150.40 | 150.47 | 0.43 | 0.29% | 0 |
| 150 | 0 | x | 149.03 | 150.84 | 151.45 | 150.44 | 1.26 | 0.84% | 0.02 |
| pmol/L | 500 | | 112.45 | 112.67 | 112.14 | 112.42 | 0.27 | 0.24% | 25.29 |
| F1 + 2 | 500 | x | 141.51 | 143.36 | 139.35 | 141.40 | 2.01 | 1.42% | 6.02 |
| | 1000 | | 77.18 | 77.64 | 76.35 | 77.06 | 0.65 | 0.85% | 48.79 |
| | 1000 | x | 141.09 | 144.19 | 141.69 | 142.33 | 1.65 | 1.16% | 5.41 |
| | 2000 | | 2.11 | 1.83 | 1.65 | 1.86 | 0.24 | 12.63% | 98.76 |
| | 2000 | x | 140.20 | 141.80 | 140.04 | 140.68 | 0.97 | 0.69% | 6.51 |
| Sample, | 0 | | 1691.02 | 1689.56 | 1678.51 | 1686.4 | 6.84 | 0.41% | 0 |
| 1700 | 0 | x | 1695.07 | 1693.90 | 1687.77 | 1692.2 | 3.92 | 0.23% | 0.35 |
| pmol/L | 500 | | 1242.68 | 1348.70 | 1352.22 | 1314.5 | 62.2 | 4.74% | 22.05 |
| F1 + 2 | 500 | x | 1592.45 | 1587.15 | 1596.36 | 1592.0 | 4.62 | 0.29% | 5.60 |
| | 1000 | | 634.64 | 650.35 | 625.00 | 636.7 | 12.8 | 2.01% | 62.25 |
| | 1000 | x | 1523.02 | 1534.10 | 1539.64 | 1532.3 | 8.46 | 0.55% | 9.14 |
| | 2000 | | 112.74 | 123.31 | 112.22 | 116.1 | 6.26 | 5.39% | 93.12 |
| | 2000 | x | 1539.09 | 1518.72 | 1544.84 | 1534.2 | 13.7 | 0.89% | 9.02 |
| Sample, | 0 | | 2589.00 | 2607.00 | 2597.00 | 2598.0 | 9.02 | 0.35% | 0 |
| 2600 | 0 | x | 2555.00 | 2648.00 | 2598.00 | 2601.0 | 46.54 | 1.79% | 0.12 |
| pmol/L | 500 | | 2344.00 | 2426.00 | 2389.00 | 2386.1 | 41.06 | 1.72% | 8.15 |
| F1 + 2 | 500 | x | 2487.00 | 2544.00 | 2538.00 | 2523.0 | 31.32 | 1.24% | 2.89 |
| | 1000 | | 1841.00 | 1890.00 | 1875.00 | 1869.0 | 25.11 | 1.34% | 28.06 |
| | 1000 | x | 2540.00 | 2490.00 | 2542.00 | 2524.0 | 29.46 | 1.17% | 2.85 |
| | 2000 | | 128.00 | 115.00 | 118.00 | 120.3 | 6.81 | 5.66% | 95.37 |
| | 2000 | x | 2400.00 | 2466.00 | 2428.00 | 2431.3 | 33.13 | 1.36% | 6.42 |

### Example 2: Neutralization of Biotin Interference in Accordance with the Invention in a D-Dimer Immunoassay by Sample Pretreatment Using Activated Carbon Particles in a Pipette Tip

To determine biotin interference, samples of three pools of citrate plasma were spiked with 80, 400 and 7200 μg/1 FEU (fibrinogen equivalent units) D-dimer with, in each case, 500, 1000 or 2000 ng/mL biotin (Sigma) and assayed on the Siemens Healthineers Atellica COAG360 coagulation analyzer using the INNOVANCE LOCI D-dimer assay (Siemens Healthineers), as described in the package insert. The INNOVANCE LOCI D-dimer assay comprises, inter alia, a first reagent containing a biotinylated first antibody having specificity for D-dimer (biotinylated assay component), a second reagent containing a second antibody having specificity for a further D-dimer epitope, the second antibody being coupled to Chemibeads, and a third reagent containing streptavidin-coated Sensibeads (biotin-binding assay component). The reagents are mixed with the sample. In the reaction, the streptavidin-coated Sensibeads bind, via the biotin-streptavidin interaction, to the biotinylated antibody having specificity for D-dimer. If D-dimer is present in the sample, there is binding of the biotinylated first antibody and also of the Chemibead-bound second antibody. In this way, the Chemibeads and the Sensibeads are brought into physical proximity with one another, so that a chemiluminescence signal is formed and can be measured. A D-dimer standard curve is used for quantification.

To neutralize biotin interference, 250 μL of sample in each case were pretreated by pipetting up and down three times using a pipette tip containing activated carbon and a separation filter, before the reagents for detection of D-dimer were mixed with the sample. For this purpose, a highly porous separation filter (PE porous, from KIK Kunststofftechnik) was inserted into a pipette tip to create a first compartment connected to the pipetting opening of the pipette tip and a second compartment separated from the pipetting opening by the separation filter, and the second compartment was filled with mg of spherical activated carbon having an average particle size of approx. 1 mm (“NorBd” Spherical Activated Carbon, Norm Technologies). The separation filter was permeable for the plasma material, but impermeable for the activated carbon.

Each sample was assayed in duplicate, and the mean of the measured D-dimer concentrations was calculated.

Table 2 shows the biotin interference in the INNOVANCE LOCI D-dimer assay in samples containing 0-2000 ng/mL biotin and the elimination of the biotin interference by the pretreatment of the samples with activated carbon. The described technique shows 100%-91.1% recovery of the treated samples in the INNOVANCE LOCI D-dimer assay for samples containing up to 2000 ng/mL biotin. In the absence of pretreatment, an increasing deviation from the starting value becomes apparent with increasing content of biotin in the sample. Just 500 ng/mL biotin lead to a deviation of up to 12% in relation to the starting value of the sample without biotin. Contents of 1000 ng/mL and 2000 ng/mL biotin in the sample lead to a deviation of up to and 94.32% from the starting value of the sample without biotin.

TABLE 2

| | | | | | | Deviation |
|---|---|---|---|---|---|---|
| | | Activated | | | | from |
| | Biotin | carbon | Measurement 1 | Measurement 2 | Mean | starting |
| Sample | [ng/ml] | added | [μg/L FEU] | [μg/L FEU] | [μg/L FEU] | value in % |
| Sample, | 0 | | 83.2 | 82.9 | 83.1 | 0 |
| 80 | 0 | X | 81.7 | 83.1 | 82.4 | 0.84 |
| μg/l | 500 | | 76.1 | 75.4 | 75.8 | 8.78 |
| FEU | 500 | X | 81.0 | 80.8 | 80.9 | 2.65 |
| | 1000 | | 57.2 | 61.3 | 59.3 | 28.65 |
| | 1000 | X | 81.4 | 79.1 | 80.3 | 3.37 |
| | 2000 | | 13.1 | 8.0 | 11.1 | 86.64 |
| | 2000 | X | 78.5 | 80.0 | 79.3 | 4.57 |
| Sample, | 0 | | 388.1 | 393.7 | 390.9 | 0 |
| 400 | 0 | X | 381.1 | 382.9 | 382.0 | 2.28 |
| μg/l | 500 | | 346.5 | 341.3 | 343.9 | 12.02 |
| FEU | 500 | X | 375.2 | 365.0 | 370.1 | 5.32 |
| | 1000 | | 205.6 | 218.8 | 212.2 | 45.71 |
| | 1000 | X | 380.7 | 364.2 | 372.7 | 4.66 |
| | 2000 | | 24.2 | 27.8 | 26.0 | 93.35 |
| | 2000 | X | 364.7 | 347.5 | 356.1 | 8.9 |
| Sample, | 0 | | 7156.4 | 7284.2 | 7220.3 | 0 |
| 7200 | 0 | X | 7205.9 | 7232.3 | 7219.1 | 0.02 |
| μg/l | 500 | | 6736.4 | 6723.2 | 6729.8 | 6.79 |
| FEU | 500 | X | 6950.6 | 6991.8 | 6971.2 | 3.45 |
| | 1000 | | 4281.5 | 4358.5 | 4335.0 | 39.96 |
| | 1000 | X | 6901.1 | 6911.0 | 6906.1 | 4.35 |
| | 2000 | | 388.4 | 432.2 | 410.3 | 94.32 |
| | 2000 | X | 6778.7 | 6693.3 | 6736.0 | 6.7 |

What is claimed is:

1. A method for neutralizing biotin interference in a binding assay for detection of an analyte in a sample, wherein the binding assay comprises contacting the sample at least with a biotinylated component and with a biotin-binding component,
wherein the sample is contacted with an adsorbent substance before or during the contacting with the biotinylated component and with the biotin-binding component,
wherein the adsorbent substance has a porous structure having a total surface area (S_{Bet}) of from 300 to 6500 m² g⁻¹ and has at least macropores, mesopores and micropores, and
wherein the volume of the micropores (V_{mic}) is from 0.15 to 0.75 cm³ g⁻¹.

2. The method as claimed in claim 1, wherein the adsorbent substance is activated carbon, a metal-organic framework (MOF), zeolite, clay, a porous organic polymer (POP), hydrotalcite, an organic-inorganic hybrid, a porous metal oxide, a lithium zirconate or a mesoporous silica material (MSM).

3. The method as claimed in claim 1, wherein the sample is contacted with the adsorbent substance by mixing the sample with the substance in the form of a powder, granular material or microfiber mixture to form a suspension.

4. The method as claimed in claim 3, wherein the sample is removed from the suspension of the adsorbent substance before the subsequent contacting with the biotinylated component and with the biotin-binding component.

5. The method as claimed in claim 1, wherein the sample is contacted with the adsorbent substance by contacting the sample with a solid phase coated with the substance.

6. The method as claimed in claim 5, wherein the solid phase coated with the adsorbent substance is a filter, surface of a liquid container, pipette tip, or particle composed of latex, Sepharose, agarose, plastic, glass, protein, alginate, silicate or metal.

7. The method as claimed in claim 1, wherein the adsorbent substance has been pretreated with one or more blocking proteins or blocking peptides.

8. The method as claimed in claim 7, wherein the one or more blocking proteins are dextran, albumin, polygeline or milk protein.

9. The method as claimed in claim 1, wherein the adsorbent substance is provided in the form of polymer-coated substance beads having a pore size with permeability for biomolecules of a mass <40 kDa.

10. The method as claimed in claim 1, wherein the biotin-binding component contains biotin-binding avidin, streptavidin, tamavidin 1 or 2, shwanavidin, rhizavidin, bradavidin, burkavidin, zebavidin, xenavidin and/or strongavidin.

11. The method as claimed in claim 1, wherein the biotin-binding component and/or the biotinylated component contains a peptide, a protein, an antibody, an antibody fragment, an affimer or an aptamer.

12. The method as claimed in claim 1, wherein the biotinylated component contains a first component of a signal-forming system and the biotin-binding component contains a second component of the signal-forming system, and wherein the first and the second component of the signal-forming system interact in such a way that a detectable signal is formed if the first and the second component of the signal-forming system are brought into physical proximity with one another.

13. The method as claimed in claim 12, wherein the first component of the signal-forming system is a chemiluminescent agent and the second component of the signal-forming system is a photosensitizer or vice versa.

14. The method as claimed in claim 1, wherein the sample is a body fluid sample from a human or an animal.

15. The method as claimed in claim 14, wherein the body fluid sample is whole blood, blood plasma, blood serum, urine, cerebrospinal fluid or lacrimal fluid.

16. A coated solid-phase system for neutralizing biotin interference in a binding assay for detection of an analyte in a sample, wherein:
(i) the coating comprises an adsorbent substance, wherein the substance has a porous structure having a total surface area (S_{Bet}) of from 300 to 6500 m² g⁻¹ and has at least macropores, mesopores and micropores, wherein the volume of the micropores (V_{mic}) is from 0.15 to 0.75 cm³ g⁻¹, wherein the substance is preferably activated carbon, a metal-organic framework (MOF), zeolite, clay, a porous organic polymer (POP), hydrotalcite, an organic-inorganic hybrid, a porous metal oxide, a lithium zirconate or a mesoporous silica material (MSM); and
(ii) the solid phase is a filter, surface of a liquid container, pipette tip, or particle composed of latex, Sepharose, agarose, plastic, glass, protein, alginate, silicate or metal.
